(19) United States
(12) Patent Application Publication
(10) Pub. No.: US 2015/0240225 A1
(43) Pub. Date: Aug. 27, 2015

(54) BIOCHEMICAL STRESS RESISTANT MICROBIAL ORGANISM

(71) Applicant: BIOCHEMTEX S.P.A., TORTONA (Alessandria) (IT)

(72) Inventors: Paola Branduardi, Milano (IT); Danilo Porro, Erba (IT)

(73) Assignee: Biochemtex S.p.A., Tortona (IT)

(21) Appl. No.: 14/431,190

(22) PCT Filed: Oct. 4, 2013
(86) PCT No.: PCT/EP2013/070739

§ 371 Date: Mar. 25, 2015

(30) Foreign Application Priority Data
Oct. 5, 2012 (IT) TO2012A000870

(51) Int. Cl. C12N 9/88; C12P 7/10

(52) U.S. Cl. CPC C12N 9/88; C12P 7/10; C12Y 403/01023; C12Y 403/01024; C12Y 403/01025; C12Y 403/01003

(57) ABSTRACT

It is disclosed a non-naturally occurring microbial organism comprising at least one exogenous nucleic acid encoding an enzyme, or a portion thereof, selected from the group of ammonia lyase. Preferably, the enzyme is PAL3, and the at least one exogenous nucleic acid is obtained from Arabidopsis thaliana. The non-naturally occurring microbial organism has an increased resistance to biochemical stress compared to the starting microbial organism, as induced for instance by oxidative stress or organic acid stress. Preferably, the non-naturally occurring microbial organism is a yeast and it may be used for fermenting a carbon source obtained from a ligno-cellulosic feedstock.

### BACKGROUND

Microbial organisms can be easily grown on an industrial scale and are widely used in the production of biochemical, such as biofuels, bio-oils, organic acids. Among used microorganisms Escherichia coli and the yeasts, in particular Saccharomyces cerevisiae are often used. However, in an industrial process, wherein the organism is used as a means for production, biochemical stress on the organism typically decreases the production of the product and/or the reproduction of the microorganism. Bacteria, yeasts, other fungi, cultured animal cells, and cultured plant cells show similar responses to biochemical stress. Biochemical stress may be caused by unwanted compounds which are formed as by-products, such as acetic acid and other organic acids, may be caused also by the accumulation of the final product, such as ethanol, or may be caused by compounds present in a starting materials used, for example, in the production of biochemicals. Microorganisms are sensitive to many others inhibitory compounds.

Different strategies have been developed for mitigating the effects of biochemical stress on microbial organisms.

WO2006113147 discloses a method for increasing stress tolerance in organisms used for industrial production. More particularly, it is disclosed a process for making L-ascorbic acid available to organisms during industrial production. The method comprises functionally transforming a recombinant organism with a coding region encoding a mannose epimerase (ME), a coding region encoding an L-galactose dehydrogenase (LGDH), and a coding region encoding a D-arabinono-1,4-lactone oxidase (ALO), whereby the recombinant organism is enabled to produce ascorbic acid endogenously.

Ammonia lyase enzymes are a well-known category of enzymes. Among ammonia lyase enzymes, phenylalanine ammonia lyase (PAL) has been widely studied. A multi-gene family usually encodes PAL. The enzyme is widely distributed in higher plants (such as Arabidopsis thaliana). Among microorganisms, PAL occurs in some fungi and abundantly in yeasts, especially in the yeast family Rhodotorula. Sporobolomyces roseus and Sporidiobolus pararoseus are also PAL-producing yeasts. Even if present in some microorganisms as an endogenous enzyme, in many cases PAL enzymes in microbial organism have a low, or very low, sequence identity with PAL enzymes present in plants. In other cases, activity of endogenous PAL enzymes in microbial organism is different from the activity of PAL enzymes in plant.

An overview of the properties of phenylalanine ammonia lyase may be found in M. Jason MacDonald and Godwin B. D'Cunha, Biochem. Cell Biol. 85: 273-282 (2007).

PAL is the first and key enzyme of the phenyl propanoid sequence. Specifically, PAL catalyses the non-oxidative deamination of phenylalanine to trans-cinnamic acid and ammonia. Cinnamic acid and derivatives provide plants with a natural protection against infections by pathogenic microorganisms.

In Alexandra Chambel et al., “Effect of cinnamic acid on the growth and on plasma membrane H⁺-ATPase activity of Saccharomyces cerevisiae”, International Journal of Food Microbiology, 50, (1999), p. 173-179, it has been proved that yeast cells grown in the presence of cinnamic acid exhibit a more active plasma membrane H⁺-ATPase. A more active H⁺-ATPase could prevent the reducing of the intracellular pH value determined by the diffusion of undissociated organic acids.

WO2008153890 discloses a method for increasing tolerance in yeast to organic acids and low pH comprising functionally transforming a yeast with at least one copy of a nucleotide sequence encoding a plasma membrane H⁺-ATPase. The present invention relates generally to the field of increasing tolerance in yeast to organic acids present in culture medium, and to low pH of the medium. More specifically, it relates to increasing H⁺-ATPase levels in yeast used in industrial production.

U.S. Pat. No. 6,521,748 discloses a genetically engineered biocatalyst possessing enhanced tyrosine ammonia-lyase activity. In particular, the patent describes methods for bioproduction of para-hydroxycinnamic acid (PHCA) through conversion of: cinnamate to PHCA; glucose to phenylalanine to PHCA via the PAL route; and through generation of a new biocatalyst possessing enhanced tyrosine ammonia-lyase (TAL) activity. The evolution of TAL requires isolation of a yeast PAL gene, mutagenesis and evolution of the PAL coding sequence, and selection of variants with improved TAL activity. The invention demonstrates the bioproduction of PHCA from glucose through the above mentioned routes in various fungi and bacteria. No increase in biochemical stress resistance of the modified microorganism is disclosed.

### BRIEF DESCRIPTION OF INVENTION

It is disclosed a non-naturally occurring microbial organism, derived from a starting microbial organism, wherein the non-naturally occurring microbial organism comprises at least one exogenous nucleic acid encoding an enzyme, or a portion thereof, selected from the group of ammonia lyase, and the non-naturally occurring microbial organism has an increased resistance to biochemical stress compared to the starting microbial organism.

It is also disclosed that the enzyme may be selected from the group consisting of EC.4.3.1.X, where X is an integer from 1 to 27 inclusive.

It is further disclosed that the enzyme may be selected from the group consisting of EC.4.3.1.23, EC.4.3.1.24, EC.4.3.1.25, and EC.4.3.1.3.

It is also disclosed that the enzyme may be selected from the group consisting of phenylalanine ammonia-lyase (PAL), tyrosine ammonia-lyase (TAL), phenylalanine/tyrosine ammonia-lyase (PAL/TAL), and histidine ammonia-lyase (HAL), preferably PAL, more preferably PAL3.

It is further disclosed that the at least one exogenous nucleic acid may be obtained from a plant, preferably from Arabidopsis thaliana.

It is also disclosed that the enzyme may comprise SEQ ID NO: 1 or an amino acid sequence having a sequence identity to SEQ ID NO: 1 selected from the group consisting of at least 98%, at least 95%, at least 90%, at least 80%, at least 70%, at least 60%, at least 50%, and at least 40%.

It is further disclosed that the enzyme may be SEQ ID NO: 2 or an amino acid sequence having a sequence identity to SEQ ID NO: 2 selected from the group consisting of at least 98%, at least 95%, at least 90%, at least 80%, at least 70%, at least 60%, at least 50%, and at least 40%.

It is also disclosed that the enzyme may be a fragment of SEQ ID NO: 2.

It is further disclosed that the biochemical stress may be induced by at least one of a reactive oxygen species, an organic acid, a mineral acid, or an alcohol.

It is also disclosed that the increased resistance to biochemical stress compared to the starting microbial organism may be indicated by at least one of an increase in the percentage of viable cells or a decrease in the amount of reactive oxygen species (ROS), under the same conditions.

It is further disclosed that the stress may be induced by ethanol and/or acetic acid.

It is also disclosed that the enzyme may catalyze a reaction to produce at least a cinnamic acid.

It is further disclosed that the at least a cinnamic acid may be selected from the group consisting of cis-cinnamic acid, trans-cinnamic acid and para-hydroxy cinnamic acid.

It is also disclosed that the at least a cinnamic acid may comprise cis-cinnamic acid.

It is further disclosed that the at least a cinnamic acid may comprise trans-cinnamic acid.

It is also disclosed that the at least a cinnamic acid may comprise parahydroxy cinnamic acid.

It is further disclosed that the starting microbial organism may be a non-naturally occurring microbial organism.

It is also disclosed that the starting microbial organism may be selected from the group consisting of yeasts, bacteria, fungi.

It is further disclosed that the starting microbial organism may be a yeast, and that preferably the yeast is selected from the group consisting of Saccharomyces, Candida, Zygosaccharomyces, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, and Schwanniomyces, and that more preferably the yeast is a Saccharomyces cerevisiae yeast, and that even more preferably the strain of Saccharomyces cerevisiae yeast is selected from the group consisting of GRF18U, BY4742, CEN.PK strains 102-5B and 113-11C, VIN13, and AP.

It is also disclosed that the starting microbial organism may be a fungus.

It is further disclosed that the starting microbial organism may be a bacterium, and that preferably the bacterium is selected from the group consisting of Escherichia coli, Klebsiella oxytoca, Anaerobiospirillum succiniciprodiicens, Actino bacillus succinogenes, Mannheimia succiniciprodiicens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida.

It is also disclosed a method for producing a product, comprising culturing the disclosed non-naturally occurring microbial organism in a culture medium comprising a carbon source, under conditions and for a sufficient period of time to produce the product, and that preferably the non-naturally occurring microbial organism is cultured in the presence of at least one biochemical stress agent.

It is further disclosed that the at least one biochemical stress agent may be selected from the group consisting of acetic acid, formic acid, hydrogen peroxide, and ethanol.

It is also disclosed that the at least one biochemical stress agent may comprise acetic acid, and that preferably the concentration of acetic acid is selected from the group consisting of higher than 3.5 g/l, higher than 7 g/l, higher than 11 g/l, and higher than 15 g/l.

It is further disclosed that the at least a fraction of the carbon source may be obtained from a ligno-cellulosic feedstock, and that preferably the ligno-cellulosic feedstock is subjected to a pretreatment to produce a pretreated ligno-cellulosic feedstock.

It is also disclosed that at least a fraction of the pretreated ligno-cellulosic feedstock is subjected to a hydrolysis process, and that preferably the hydrolysis process is an enzymatic hydrolysis.

It is further disclosed that the product comprises ethanol.

### BRIEF DESCRIPTION OF FIGURES

FIG. 1 is a schematic representation of the different subpopulations obtained by flow cytometry.

FIG. 2 is the amino-acidic sequence SEQ ID NO: 1 according to one embodiment of the present invention.

FIG. 3 is the amino-acidic sequence SEQ ID NO: 2 according to another embodiment of the present invention.

FIG. 4 is the schematic representation of the screening procedure used in the experiments.

FIG. 5 is the graph of the growth of transformants in minimal medium and in the presence of hydrogen peroxide.

FIG. 6 is the graph of the growth of transformants in minimal medium and in the presence of oxidative and acidic stress.

FIG. 7 is the graph of cytofluorimetric analysis for three strains.

FIG. 8 is the graph of growth kinetic of wild type strains, strains transformed with the sole control plasmid, and the strains bearing the total AtPAL3 coding sequence in the presence of acetic acid for S. cerevisiae strains VIN13 and AP.

FIG. 9 is the graph of glucose concentration and ethanol concentration of the modified and the not modified AP strain.

### DETAILED DESCRIPTION

It is disclosed a non-naturally occurring microbial organism derived from a starting microbial organism, having an increased resistance to biochemical stress compared to the starting microbial organism. The non-naturally occurring microbial organism comprises at least one exogenous nucleic acid encoding an enzyme of the group of ammonia lyases, or a portion of the enzyme. By a portion of an enzyme it is meant a portion of the amino-acidic sequence encoding the enzyme.

In the present specification, the terms “microbial,” “microbial organism” or “microorganism” are equivalent terms for indicating any organism that exists as a microscopic cell included within the domains of archaea, bacteria or eukarya. Therefore, the term comprises prokaryotic or eukaryotic cells or organisms having a microscopic size and includes bacteria, archaea and eubacteria of all species as well as eukaryotic microorganisms such as yeasts and fungi. The term also includes cells of any species that can be cultured for the production of a biochemical.

The term “non-naturally occurring” microbial organism or microorganism of the invention means that the microbial organism has at least one genetic alteration not normally found in a naturally occurring strain of the referenced species. Genetic alterations include, for example, modifications introducing expressible nucleic acids encoding polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

The present invention discloses genetic alterations that can be designed and inserted in a starting microbial organism to increase the resistance to biochemical stress. These non-naturally occurring microbial organisms also can be subjected to adaptive evolution to further increase the resistance to biochemical stress.

“Exogenous” as it is used herein is intended to mean that the referenced molecule or the referenced activity is introduced into the starting microbial organism. The molecule can be introduced, for example, by introduction of an encoding nucleic acid into the genetic material of the starting microbial organism such as by integration into a starting microbial organism chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to an enzymatic or biosynthetic activity, the term refers to an activity that is introduced into the reference starting microbial organism. The source can be, for example, a homologous or heterologous encoding nucleic acid that expresses the referenced activity following introduction into the host microbial organism. Therefore, the term “endogenous” refers to a referenced molecule or activity that is present in the starting microbial organism. Similarly, the term when used in reference to expression of an encoding nucleic acid refers to expression of an encoding nucleic acid contained within the starting microbial organism. The term “heterologous” refers to a molecule or activity derived from a source other than the referenced species whereas “homologous” refers to a molecule or activity derived from the starting microbial organism. Accordingly, exogenous expression of an encoding nucleic acid of the invention can utilize either or both a heterologous or homologous encoding nucleic acid.

When more than one exogenous nucleic acid is included in a starting microbial organism, it is intended to mean that the referenced molecules or the referenced activities are introduced into the starting microbial organism, as discussed above. It is further understood, as disclosed herein, that such more than one exogenous nucleic acids can be introduced into the starting microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a starting microbial organism, it is understood that the two exogenous nucleic acids can be introduced as a single nucleic acid, for example, on a single plasmid, on separate plasmids, can be integrated into the chromosome of the starting microbial organism at a single site or multiple sites, and still be considered as two exogenous nucleic acids. Similarly, it is understood that more than two exogenous nucleic acids can be introduced into a starting microbial organism in any desired combination, for example, on a single plasmid, on separate plasmids, can be integrated into the chromosome of the starting microbial organism at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

The non-naturally occurring microbial organisms of the invention can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Such genetic alterations include, for example, genetic alterations of species homologs, in general, and in particular, orthologs, paralogs or nonorthologous gene displacements.

An ortholog is a gene or genes that are related by vertical descent and are responsible for substantially the same or identical functions in different organisms. Genes are related by vertical descent when, for example, they share sequence similarity of sufficient amount to indicate they are homologous, or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Genes that are orthologous can encode proteins with sequence similarity of about 25% to 100% amino acid sequence identity. Genes encoding proteins sharing an amino acid similarity less that 25% can also be considered to have arisen by vertical descent if their three-dimensional structure also shows similarities.

Orthologs include genes or their encoded gene products that through, for example, evolution, have diverged in structure or overall activity. For example, where one species encodes a gene product exhibiting two functions and where such functions have been separated into distinct genes in a second species, the three genes and their corresponding products are considered to be orthologs. For the production of a biochemical product, the orthologous gene harboring the metabolic activity to be introduced or disrupted is to be chosen for construction of the non-naturally occurring microorganism.

In contrast, paralogs are homologs related by, for example, duplication followed by evolutionary divergence and they have similar or common, but not identical functions. Paralogs can originate or derive from, for example, the same species or from a different species. Paralogs are proteins from the same species with significant sequence similarity to each other suggesting that they are homologous, or related through co-evolution from a common ancestor.

A nonorthologous gene displacement is a nonorthologous gene from one species that can substitute for a referenced gene function in a different species. Substitution includes, for example, being able to perform substantially the same or a similar function in the species of origin compared to the referenced function in the different species. Although generally, a nonorthologous gene displacement will be identifiable as structurally related to a known gene encoding the referenced function, less structurally related but functionally similar genes and their corresponding gene products nevertheless will still fall within the meaning of the term as it is used herein. Functional similarity requires, for example, at least some structural similarity in the active site or binding region of a nonorthologous gene product compared to a gene encoding the function sought to be substituted. Therefore, a nonorthologous gene includes, for example, a paralog or an unrelated gene.

The starting microbial organism can be selected both from naturally occurring, and non-naturally occurring microbial organisms generated in, for example, bacteria, yeasts, fungi or any of a variety of other microorganisms applicable to fermentation. By fermentation it is meant the conversion of a carbon source to products. Exemplary bacteria include species selected from Escherichia coli, Klebsiella oxytoca, Anaerobiospirillum succiniciprodiicens, Actino bacillus succinogenes, Mannheimia succiniciprodiicens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. E. coli is a particularly useful starting microbial organism since it is a well characterized microbial organism suitable for genetic engineering.

Other particularly useful host organisms include yeasts. The yeast can be selected from any known genus and species of yeasts. Yeasts are described for example by N. J. W. Kreger-van Rij, “The Yeasts,” Vol. 1 of Biology of Yeasts, Ch. 2, A. H. Rose and J. S. Harrison, Eds. Academic Press, London, 1987. The yeast genus may be Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, or Schwanniomyces, among others. Preferably the yeast is selected from Saccharomyces. More preferably the yeast is S. cerevisiae. In a particularly preferred embodiment, the yeast is S. cerevisiae strain GRF18U, BY4742 (EuroScarf Accession No. Y10000), CEN.PK strains 102-5B (MATa, ura3-52, his3-11, leu2-3/112, TRP1, MAL2-8c, SUC2) and 113-11C (MATa, ura3-52, his3-11, TRP1, MAL2-8c, SUC2—Dr. P. Kötter, Institute of Microbiology, Johann Wolfgang Goethe-University, Frankfurt, Germany), VIN13, AP.

The yeast may be haploid or diploid.

It is understood that any suitable starting microbial organism can be used to introduce metabolic and/or genetic modifications to produce a non-naturally occurring microbial organism having an increased resistance to biochemical stress.

The exogenous nucleic acid encoding the enzyme of the present disclosure can be obtained, or isolated, or extracted, from any source, comprising bacteria, prokaryotes, eukaryotes, microorganisms, fungi, plants, or animals. The exogenous nucleic acid may be synthesized by chemical means. Preferably, the exogenous nucleic acid is isolated from a plant. In an even more preferred embodiment, the exogenous nucleic acid encoding the disclosed enzyme is obtained from Arabidopsis thaliana. It should be noted that a nucleic acid is “isolated” from an organism if it encodes a protein sequence substantially identical to the protein encoded in the starting microbial organism.

Genetic material comprising the exogenous nucleic acid can be extracted from cells of the organism by any known technique. Thereafter, the coding region can be isolated by any appropriate technique. In one known technique, the exogenous nucleic acid is isolated by, first, preparing a genomic DNA library or a cDNA library, and second, identifying the exogenous nucleic acid in the genomic DNA library or cDNA library, such as by probing the library with a labelled nucleotide probe selected to be or presumed to be at least partially homologous with the exogenous nucleic acid, determining whether expression of the exogenous nucleic acid imparts a detectable phenotype to a library microorganism comprising the exogenous nucleic acid, or amplifying the desired sequence by PCR. Other known techniques for isolating the coding region can also be used. It should be noted that in this context “exogenous nucleic acid” refers to a nucleic acid which is exogenous with respect to the starting microbial organism and not to the cells from which it is extracted. Namely, the “exogenous nucleic acid” may be exogenous or endogenous with respect to the cell from which it is extracted.

Preferably, the exogenous nucleic acid encoding the disclosed enzyme is inserted into the starting microbial organism in such a manner that the original enzyme activity is produced in the non-naturally occurring microbial organism and is substantially functional. Such a non-naturally occurring microbial organism is referred to herein as being “functionally transformed” or “functionally expressed.”

Recombinant DNA techniques are well-known, such as in Sambrook et al., Molecular Genetics: A Laboratory Manual, Cold Spring Harbor Laboratory Press, which provides further information regarding various techniques known in the art and discussed herein.

Once the exogenous nucleic acid has been extracted, or isolated or synthesized, it is prepared for being incorporated in the starting microbial organism. Preferably, the exogenous nucleic acid is inserted into a vector and operably linked to a promoter found on the vector and active in the starting microbial organism. Any vector, for instance integrative, chromosomal or episomal vectors, can be used.

Any promoter active in the host microbial organism for instance homologous, heterologous constitutive, inducible or repressible promoters, can be used.

A promoter, as is known, is a DNA sequence that can direct the transcription of a nearby coding region. Constitutive promoters continually direct the transcription of a nearby coding region. Inducible promoters can be induced by the addition to the medium of an appropriate inducer molecule, which will be determined by the identity of the promoter. Repressible promoters can be repressed by the addition to the medium of an appropriate repressor molecule, which will be determined by the identity of the promoter. Preferably, the promoter is constitutive. In one preferred embodiment, the starting microbial organism is a yeast (of the genus of Saccharomyces cerevisiae) and the constitutive promoter is the S. cerevisiae triose-phosphate-isomerase (TPI) promoter.

The insertion of the exogenous nucleic acid into the vector may involve the use of restriction endonucleases to open the vector in a suitable position where operable linkage to the promoter is possible, followed by ligation of the exogenous nucleic acid at that position. Before insertion into the vector, the exogenous nucleic acid may be prepared for use in the starting microbial organism. Among possible preparation techniques known in the art, this may involve altering the codons used in the exogenous nucleic acid to more fully match the codon use of the starting microbial organism; changing sequences in the exogenous nucleic acid that could impair the transcription or translation of the exogenous nucleic acid or the stability of an mRNA transcript of the exogenous nucleic acid; or adding or removing portions encoding signalling peptides, which are regions of the protein encoded by the exogenous nucleic acid that direct the protein to specific locations (e.g. an organelle, the membrane of the cell or an organelle, or extracellular secretion).

The vector comprising the exogenous nucleic acid operably linked to the promoter may be a plasmid, a cosmid, or an artificial chromosome of the starting microbial organism, among others known in the art. In addition to the exogenous nucleic acid operably linked to the promoter, the vector may also comprise other genetic elements. For example, if the vector is not expected to integrate into the genome of the starting microbial organism, the vector may comprise an origin of replication, which allows the vector to be passed on to progeny cells of the starting microbial organism comprising the vector (e.g. 2μ derived or ARS/CEN). If integration of the vector into the genome of the starting microbial organism is desired, the vector may comprise sequences homologous to sequences found in the genome of the starting microbial organism, and may also comprise coding regions that can facilitate integration. To determine which cells of the starting microbial organism are transformed, the vector may comprise a selectable marker or screenable marker which imparts a phenotype to the transformed microbial organism that distinguishes it from the starting microbial organism. For instance, the e.g. the transformed microbial organism may survive in a medium comprising an antibiotic fatal to the starting microbial organism or it can metabolize a component of the medium into a product that the starting microbial organism does not, among other phenotypes. In addition, the vector may comprise other genetic elements, such as restriction endonuclease sites and others typically found in vectors.

After the vector is prepared, with the exogenous nucleic acid operably linked to the promoter, the starting microbial organism may be transformed with the vector (i.e. the vector can be introduced into at least one of the cells of a population of the starting microbial organism). Techniques for microbial organism transformation are well established, and include electroporation, micro-projectile bombardment, and the LiAc/ssDNA/PEG method, among others. Transformed microbial organisms may then be detected by the use of a screenable or selectable marker on the vector. It should be noted that the phrase “transformed microbial organism” has essentially the same meaning as “non-naturally occurring microbial organism”. The transformed microbial organism can be one that received the vector in a transformation technique, or can be a progeny of such transformed microbial organism.

According to the present disclosure, the non-naturally occurring microbial organism comprises at least one exogenous nucleic acid encoding an enzyme selected from the group of ammonia lyases.

Ammonia lyases are a class of enzyme which removes ammonia or amino groups by cleaving at least one ammonia C-N bond in a substrate, thereby leaving a C═C double carbon bonds.

In the Enzyme Commission classification, ammonia lyases are classified as EC.4.3.1.X, where X is an integer from 1 to 27 inclusive. It is remarked that the Enzyme Commission number is a numerical classification scheme for enzymes, based on the chemical reactions they catalyze. Thereby, EC numbers do not specify enzymes, but enzyme-catalyzed reactions. If different enzymes (for instance from different organisms) catalyze the same reaction, then they receive the same EC number.

Preferably, the enzyme is selected from the aromatic amino acid lyase classes of EC.4.3.1.23, EC.4.3.1.24, EC.4.3.1.25, EC.4.3.1.3.

More preferably, the enzyme is selected from the group consisting of PAL, TAL, PAL/TAL, HAL.

A phenylalanine ammonia-lyase, also indicated as PAL, is an enzyme that catalyzes the chemical reaction:

L-phenylalanine⇄trans-cinnamate+NH₃

Hence, this enzyme acts on one substrate, L-phenylalanine, and produces two products, trans-cinnamate and ammonia.

Trans-cinnamate is the ionized form of trans-cinnamic acid, and it is understood that trans-cinnamate and cinnamic acid can be used interchangeably throughout to refer to the compound in any of its neutral or ionized forms, including any salt forms thereof. The same consideration holds for the products of the reactions catalyzed by TAL, PAL/TAL, HAL.

A tyrosine ammonia-lyase, also indicated by TAL, is an enzyme that catalyzes the chemical reaction:

L-tyrosine⇄trans-p-hydroxycinnamate+NH₃

Hence, this enzyme acts on one substrate, L-tyrosine, and produces two products, trans-p-hydroxycinnamate and ammonia.

A phenylalanine/tyrosine ammonia-lyase, also indicated as PAL/TAL, is an enzyme that catalyzes both the chemical reactions:

L-phenylalanine⇄trans-cinnamate+NH₃

L-tyrosine⇄trans-p-hydroxycinnamate+NH₃

Hence, this enzyme may acts on both substrates, L-phenylalanine and L-tyrosine.

A histidine ammonia-lyase, also indicated as HAL, is an enzyme that catalyzes the chemical reaction:

L-histidine⇄urocanate+NH₃

Hence, this enzyme acts on one substrate, L-histidine, and produces two products, urocanate and ammonia.

In a preferred embodiment, the enzyme encoded by the exogenous nucleic acid is PAL.

The disclosed non-naturally occurring microbial organism has an increased resistance to biochemical stress compared to the starting microbial organism.

By biochemical stress it is meant a stress induced on a cell by a chemical agent. Biochemical stress may be induced, for instance, by oxidative species, organic or mineral acids, or alcohols. Biochemical agents may be comprised in the cultivation medium, that is being comprised in the external environment surrounding the cell, or may be a result of the metabolic activity of the cell. Biochemical stress may be caused also by chemicals produced by the cell, for instance in the case of sugar fermentation to ethanol by means of yeasts.

Among biochemical stress, oxidative stress plays a crucial role.

Oxidative stress is defined as an imbalance in prooxidants and antioxidants, which results in macromolecular damage and disruption of redox signaling and control. It can be caused both by free radicals and by non-radical oxidants.

More in detail, biochemical stress describes cell damage caused by an overabundance of oxidants, including reactive oxygen species (ROS).

In respect to ROS, in a balanced cell state, these chemical species (e.g., oxygen ions, free radicals, and peroxide) are produced as a byproduct of respiratory metabolic processes and the level of ROS can be controlled with antioxidants, including vitamin E and vitamin C; small molecular weight peptides and cofactors, including glutathione and pyruvate; and enzymes, including superoxide dismutase and catalase.

In the term “oxidants” besides ROS other molecules are comprised, including the so-called pro-oxidant species, which can be defined as species that cause or promote oxidation.

Different weak organic acids have been described to elicit this effect (Piper et al., 1999), and among them acetic acid (Semchyshyn et al., 2011), formic acid (Du et al, 2008), lactic acid (Abbott et al., 2009). Moreover, there are evidences that also short-chain alcohols can act as pro-oxidant, as demonstrated for ethanol (Yang et al., 2012; Kim et al., 2012).

Generally, in a state of cellular imbalance, in which the levels of oxidants outweigh the levels of antioxidants, damage is caused to nuclear and mitochondrial DNA, proteins, and lipids. If this damage is irreparable, then injury, mutagenesis, carcinogenesis, accelerated senescence, and cell death can occur. In these condition cellular activities and, in turn, production and productivity may decline.

Resistance of a microbial organism to biochemical stress may be measured by means of many techniques known in the art: among others, flow cytometry is a powerful tool for interrogating the phenotype and characteristics of cells. Therefore, it facilitates the identification of different cell types within a heterogeneous population. Analysis and differentiation of the cells is based on size, granularity, and whether the cell is carrying fluorescent molecules. Indeed, flow cytometry mainly uses the principles of light scattering and emission of fluorochrome molecules to generate specific mono or multi-parameter data from particles and cells.

In flow cytometry, cells are hydro-dynamically focused in a sheath before intercepting an optimally focused light source. The cells may be labeled with fluorochrome-linked antibodies or stained with fluorescent membrane, cytoplasmic, or nuclear dyes (generically referred as fluorochromes), among others. Thus, differentiation of cell types, the presence of membrane receptors and antigens. ROS, membrane potential, membrane integrity, pH, enzyme activity, and DNA content, among others, may be detected and analyzed. Lasers are most often used as a light source in flow cytometry. When a cell intercepts the light source, it scatters light and the fluorochromes are excited to a higher energy state. This energy is released as a photon of light with specific spectral properties unique to different fluorochromes. Of course the amount of energy released can be easily quantified. One unique feature of flow cytometry is that it measures fluorescence per cell or particle. In conclusion, analysis and differentiation of the cells is mainly based on size, granularity, and whether the cell is carrying fluorescent molecules.

Flow cytometry is the preferred technique used for determining the increased resistance of a cell to biochemical stress according to the present disclosure.

The increased resistance to biochemical stress of the disclosed non-naturally microbial organism compared to the starting microbial organism may be indicated by at least one of an increase in the percentage of viable cells or a decrease in the amount of reactive oxygen species (ROS), under the same conditions. A preferred protocol for determining the resistance of a microbial organism to biochemical stress is described in details in the experimental section.

Determination of viability in microbial samples, throughout the analysis of the membrane integrity, is one of the most routine and straightforward analysis carried out by means of flow cytometry. Propidium Iodide (PI) is the most commonly used fluorescent dye for the determination of the yeast cell viability. PI is a membrane-impermeant nucleic acid stain that is excluded from viable cells. It enters cells with compromised membranes, where it binds DNA/RNAds and emits red fluorescence upon excitation. PI can be excited with an Argon laser (488 nm) and the highest emission is at 617 nm. Published evidences suggest that, under some conditions, yeast can recover from the loss of membrane integrity that allows PI to enter the cell, further proving that the yeast plasma membranes may develop a transient permeability to molecules in their environment (Haase and Reed, 2002; Davey and Hexley, 2011).

Reactive Oxygen Species, such as hydrogen peroxide, the superoxide anion and hydroxyl radicals, are normally produced through incomplete reduction of O₂ during respiration. Moreover, a variety of stressful agents, of metabolic or environmental origin, can indirectly lead to ROS generation. ROS are generally considered as key intermediates among the common stress factors and their involvement in lipid, protein and nucleic acid biochemical damages has been demonstrated. Consequences of such cellular damages include lowered metabolic activity, lowered growth rate and even decreased viability (reviewed in Kim et al., 2006 and Skulachev 2006). ROS can be easily determined by flow cytometry. ROS can be detected by dihydrorhodamine 123 (DHR123) as described in Madeo et al. (1999). Dihydrorhodamine 123 is an uncharged and nonfluorescent reactive oxygen species (ROS) indicator that can passively diffuse across membranes where it is oxidized to cationic rhodamine 123 which exhibits green fluorescence.

PI and DHR123 fluorochromes can be excited with an Argon laser (488 nm). However, the two emission spectra are quite different. The highest emission peak for the PI is at 617 nm, while the highest emission peak for DHR123 is at 534 nm. Therefore, when cells are stained with both fluorochromes, the single fluorescence related to cell viability or ROS content for any single cells can be easily quantified and discriminated.

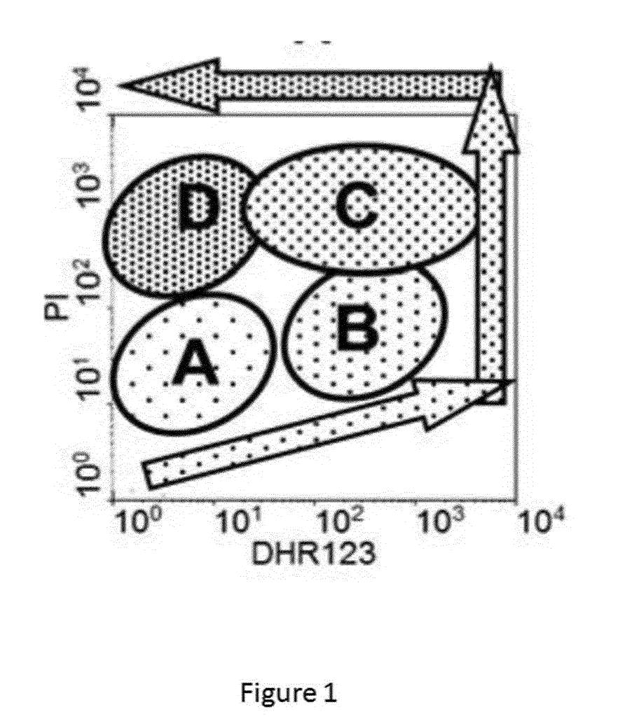

In FIG. 1 is reported a schematic representation of the different subpopulations that can be observed in dot plot treating a microbial organism population with PI and DHR123 (rhodamine signal is reported in the abscissa and PI signal on the ordinate axes).

On the dot plot (DHR123 vs. PI) each individual cell is represented by a single dot and it is quite easy to recognize at least four distinct yeast subpopulations. A first healthy subpopulation (named A), having only the background signal (autofluorescence) for both fluorochromes (low DHR and low PI signal); Indeed, like above described, flow cytometry allow the quantification of the emitted fluorescence (in this case PI and DHR123). However, any single cell emits what is so called autofluorescence (i.e., the fluorescence emitted from NOT stained cells or cells which do not stain even in the presence of the fluorochromes, like in this case). A second subpopulation (named B) of still viable but ROS accumulating cells (low PI, high rhodamine signal); a third subpopulation (named C) made of damaged cells displaying high ROS and high PI signals and finally a fourth subpopulation (named D) of dead cells, presumably originated from subpopulation C by loosing all DHR signal (Branduardi et al, 2007).

Simply to underline once more the concept, the arrows in FIG. 1 indicate the degree of cellular health, starting from the most healthy subpopulation which is of course represented by the subpopulation named A (cells that are not stained even in the presence of the fluorochromes), then comes the subpopulation named B (good viability, ROS accumulation), then the subpopulation C and finally D.

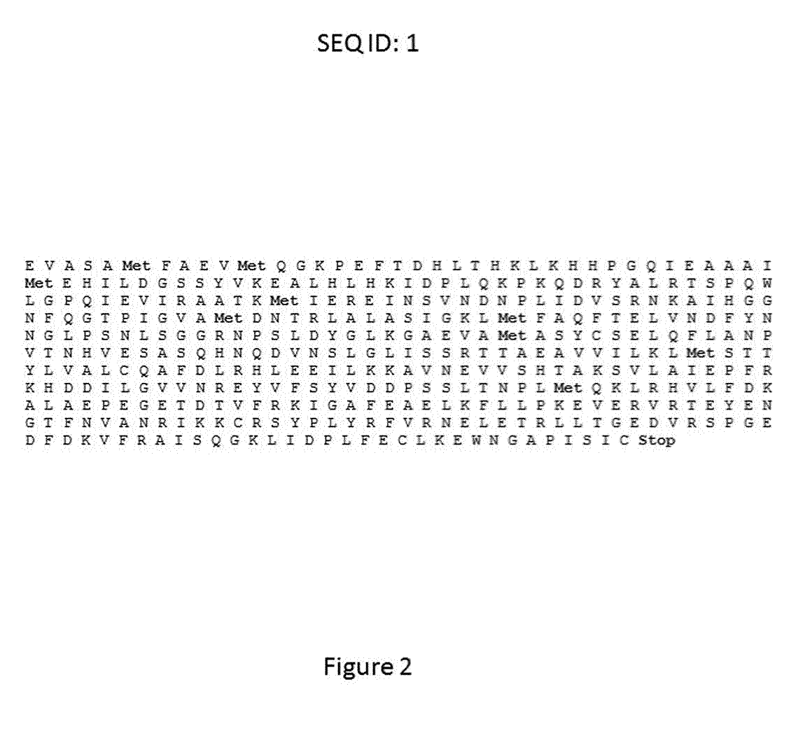

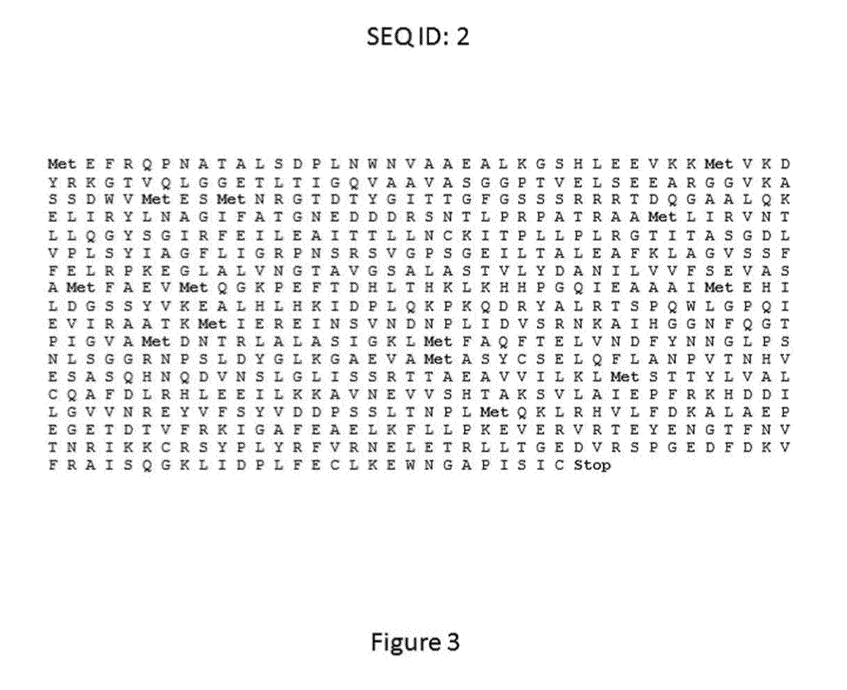

Because of the degeneracy of the genetic code, there exists a finite set of nucleotide sequences which can code for a given amino acid sequence. It is understood that all such equivalent sequences are operable variants of the disclosed sequence, since all give rise to the same protein (i.e., the same amino acid sequence) during transcription and translation, and are hence encompassed by the instant invention. Of particular interest herein are those nucleotide sequences that encode for the enzymes having the amino acid sequence represented by SEQ ID NO: 1 and SEQ ID NO: 2 and reported in FIG. 2 and FIG. 3 respectively.

Two polypeptides are said to be “identical” if the sequence of amino acid residues, in the two sequences is the same when aligned for maximum correspondence as described below.

Sequence comparisons between two (or more) polypeptides are typically performed by comparing sequences of the two sequences over a segment or “comparison window” to identify and compare local regions of sequence similarity. Optimal alignment of sequences for comparison may be conducted by the local homology algorithm of Smith and Waterman [Adv. Appl. Math. 2:482 (1980], by the homology alignment algorithm of Needleman and Wunsch [J. Mol. Biol. 48:443 (1970)], by the search for similarity method of Pearson and Lipman [Proc. Natl. Acad. Sci. (U.S.A) 85:2444 (1988)], by computerized implementations of these algorithms [(BLAST, GAP, BESTFIT, FASTA, and TFASTA in the Wisconsin Genetics Software Package, Genetics Computer Group (GCG), 575 Science Dr., Madison, Wis.], or by inspection.

“BLAST method of alignment” is an algorithm provided by the National Center for Biotechnology Information (NCBI) to compare polypeptides sequences. In the context of the present disclosure, BLAST is the reference method for comparing polypeptides sequence, by using default parameters of the BLAST software.

“Sequence identity” is determined by comparing two optimally aligned sequences over a comparison window, wherein the portion of the polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity.

The reference sequence herein is either the coding region defined by SEQ ID NO: 1 or a region of SEQ ID NO: 2 comprising the coding region. One of skill in the art will recognize that the percentage values can be appropriately adjusted to determine corresponding identity of proteins encoded by two nucleotide sequences by taking into account codon degeneracy, amino acid similarity, reading frame positioning and the like. Preferably, the enzyme encoded by the at least one exogenous nucleic acid has a sequence identity to SEQ ID NO: 1 of at least 98%, more preferably of at least 95%, even more preferably of at least 90%, yet even more preferably of at least 80%, yet even more preferably of at least 70%, yet even more preferably of at least 60%, yet even more preferably of at least 50%, being a sequence identity of at least 40% most preferred. In another embodiment, preferably the enzyme encoded by the at least one exogenous nucleic acid has a sequence identity to SEQ ID NO: 2 of at least 98%, more preferably of at least 95%, even more preferably of at least 90%, yet even more preferably of at least 80%, yet even more preferably of at least 70%, yet even more preferably of at least 60%, yet even more preferably of at least 50%, being a sequence identity of at least 40% most preferred. In another embodiment, the enzyme is a fragment of SEQ ID NO: 2.

At least a cinnamic acid may be accumulated in the non-naturally occurring microbial organism. By the expression “a product is accumulated in the non-naturally occurring microbial organism” it is meant that the product is made available inside the cell of the microbial organism.

If the product accumulated in the non-naturally occurring microbial organism is not further subjected to biochemical conversion, it may be present in the microbial organism and it can be detected and harvested.

If at least a fraction of the product accumulated in the non-naturally occurring microbial organism is further subjected to biochemical conversion reactions, not necessarily it will be present and detectable, depending on the intracellular condition, the reaction kinetics and the fraction of the product involved in the subsequent conversion.

The at least a cinnamic acid may be cis-cinnamic acid, trans-cinnamic acid or para-hydroxy cinnamic acid or a combination thereof. Preferably, the cinnamic acid comprises trans-cinnamic acid.

The disclosed non-naturally occurring microbial organism may be cultured in a culture medium comprising a carbon source. “Culturing in a culture medium” refers to the growth of a microorganism and/or the accumulation of the product produced by the microorganism in the culture medium.

The medium in which the non-naturally occurring microbial organism can be cultured can be any medium known in the art to be suitable for this purpose. Culturing techniques and media are well known in the art.

“Carbon source” refers to an organic compound (e.g., defined carbon source, such as glucose, among others) or a mixture of organic compounds (e.g., yeast extract), which can be assimilated by a microorganism (e.g., yeast) and converted to a product.

Carbon sources commonly used for culturing the non-naturally occurring microbial organism may include carbohydrates, comprising complex carbohydrates such as cellulose and hemicellulose, starch, and simple carbohydrates such as oligomeric and monomeric sugars. Oligomeric and monomeric sugars may be derived from complex carbohydrates. In the context of the present disclosure, simple sugars are the monomeric sugars, and may be selected from the group consisting of glucose, xylose, arabinose, mannose, galactose, and fructose. It should be noted that there may be other simple sugars not in the preceding list.

The non-naturally occurring microbial organism is cultured in a medium with a carbon source and other essential nutrients, under conditions and for a sufficient time to produce the product dependent on the host microbial organisms. A person skilled in the art may easily define the suitable culture conditions, according to the host microorganism needs. In an preferred embodiment, the product is ethanol and the starting microbial organism is a non-naturally occurring yeast.

In some embodiments, culture conditions include anaerobic or substantially anaerobic growth or maintenance conditions. Exemplary anaerobic conditions are well known in the art. Such conditions can be obtained, for example, by culturing the non-naturally occurring microbial organism in a fermenter which can be sealed, or isolated from the external environment, and then sparging the medium. For non-naturally occurring microbial organism where growth is not observed anaerobically, microaerobic conditions can be applied.

Nitrogen sources, growth stimulators and the like may be added to improve the microorganism cultivation and terephthalate production. Nitrogen sources include urea, ammonia salts (for example NH₄Cl or NH₄ SO4) and peptides. Protease may be used, e.g., to digest proteins to produce free amino nitrogen (FAN). Such free amino acids may function as nutrient for the host cell, thereby enhancing the growth and enzyme or enzyme mixture production. Preferred cultivation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of minerals include minerals and mineral salts that can supply nutrients comprising phosphorus, potassium, magnesium, sulphur, calcium, iron, zinc, manganese and copper.

Optionally, the pH of the medium can be maintained at a desired pH, in particular neutral pH, such as a pH of around 7 by addition of a base, such as NaOH or other bases, or acid, as needed to maintain the culture medium at a desirable pH.

Cultivation procedures are well known in the art. Briefly, cultivation can be utilized in, for example, fed-batch cultivation and batch cultivation; fed-batch cultivation and continuous separation, or continuous cultivation and continuous separation. Examples of batch and continuous cultivation procedures are well known in the art.

During the cultivation of the microorganism the carbon source is normally consumed by the microorganism and new carbon source is added. A person skilled in the art can easily determine the procedure for adding the carbon source according to the invention, for instance by monitoring carbon source depletion over time and measuring the microbial organism growth rate by measuring optical density using a spectrophotometer.

The carbon source may be added to the culture medium in a continuous, semi-continuous or single step manner. According to the invention the carbon source may be added to the culture medium either prior to inoculation, simultaneously with inoculation or after inoculation of non-naturally occurring microorganism in the culture medium.

The product produced by the non-naturally occurring microbial organism maybe removed or separated from the culture medium by any techniques known in the art and still to be invented. The removal or separation may occur in a batch, continuous, or semi-continuous manner and may involve purification processes.

The product produced by the non-naturally occurring microbial organism may be further converted to other compounds. Preferably, the conversion occurs after the product removal or separation from the culture medium. The conversion process may include chemical and biological conversion process.

Preferably, the culture of the non-naturally occurring microbial organism occurs in the presence of at least one biochemical stress agent. The biochemical stress agents may be contained in the culture medium, as a result of the preparation process of the culture medium or of some component of the culture medium, or may be contained in the carbon source. The biochemical stress agents may be, among others, acetic acid, formic acid, hydrogen peroxide, ethanol. The biochemical stress agent may be also the product produced by the non-naturally occurring microbial organism, such as ethanol or other alcohols in the case of many yeast fermentations. In the case the biochemical stress agent comprises acetic acid, preferably the concentration of acetic acid in the culture medium is higher than 3.5 g/l, more preferably higher than 7 g/l, even more preferably higher than 11 g/l, most preferably higher than 15 g/l.

In a preferred embodiment, the carbon source is obtained from a ligno-cellulosic biomass, referred to also as ligno-cellulosic feedstock.

In general, a ligno-cellulosic biomass can be described as follows:

Apart from starch, the three major constituents in plant biomass are cellulose, hemicellulose and lignin, which are commonly referred to by the generic term lignocellulose. Polysaccharide-containing biomasses as a generic term include both starch and ligno-cellulosic biomasses. Therefore, some types of feedstock can be plant biomass, polysaccharide containing biomass, and ligno-cellulosic biomass.

Polysaccharide-containing biomasses according to the present invention include any material containing polymeric sugars e.g. in the form of starch as well as refined starch, cellulose and hemicellulose.

Relevant types of biomasses for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinus sylvestris, Pinus radiate; hardwood e.g. Salix spp. Eucalyptus spp.; tubers e.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all because the characterization is primarily to the unique characteristics of the lignin and surface area.

The ligno-cellulosic biomass feedstock used to derive the composition is preferably from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins usually entire. The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath.

Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glumes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poa annua and oat). Examples of perennial cool season are orchard grass (cocksfoot, Dactylis glomerata), fescue (Festuca spp), Kentucky Bluegrass and perennial ryegrass (Lolium perenne). Examples of annual warm season are corn, sudangrass and pearl millet. Examples of Perennial Warm Season are big bluestem, indiangrass, bermuda grass and switch grass.

One classification of the grass family recognizes twelve subfamilies: These are 1) Anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed; 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae and 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding. Larger bamboos and Arundo donax have stout culms that can be used in a manner similar to timber, and grass roots stabilize the sod of sod houses. Arundo is used to make reeds for woodwind instruments, and bamboo is used for innumerable implements.

Another ligno-cellulosic biomass feedstock may be woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants.

The two main groups in which secondary xylem can be found are:

1) conifers (Coniferae): there are some six hundred species of conifers. All species have secondary xylem, which is relatively uniform in structure throughout this group. Many conifers become tall trees: the secondary xylem of such trees is marketed as softwood.

2) angiosperms (Angiospermae): there are some quarter of a million to four hundred thousand species of angiosperms. Within this group secondary xylem has not been found in the monocots (e.g. Poaceae). Many non-monocot angiosperms become trees, and the secondary xylem of these is marketed as hardwood.

The term softwood is used to describe wood from trees that belong to gymnosperms. The gymnosperms are plants with naked seeds not enclosed in an ovary. These seed “fruits” are considered more primitive than hardwoods. Softwood trees are usually evergreen, bear cones, and have needles or scale like leaves. They include conifer species e.g. pine, spruces, firs, and cedars. Wood hardness varies among the conifer species.

The term hardwood is used to describe wood from trees that belong to the angiosperm family. Angiosperms are plants with ovules enclosed for protection in an ovary. When fertilized, these ovules develop into seeds. The hardwood trees are usually broad-leaved; in temperate and boreal latitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

Therefore a preferred ligno-cellulosic biomass may be selected from the group consisting of the grasses and woods. Another preferred ligno-cellulosic biomass can be selected from the group consisting of the plants belonging to the conifers, angiosperms, Poaceae and families. Another preferred ligno-cellulosic biomass may be that biomass having at least 10% by weight of it dry matter as cellulose, or more preferably at least 5% by weight of its dry matter as cellulose.

According to the invention, ligno-cellulosic feedstock is preferably pre-treated. The term “pre-treated” may be replaced with the term “treated”. However, preferred techniques contemplated are those well known for “pre-treatment” of ligno-cellulosic biomass as will be describe further below.

As mentioned above treatment or pre-treatment may be carried out using conventional methods known in the art, which promotes the separation and/or release of cellulose and increased accessibility of the cellulose from ligno-cellulosic biomass.

Pre-treatment techniques are well known in the art and include physical, chemical, and biological pre-treatment, or any combination thereof. In preferred embodiments the pre-treatment of ligno-cellulosic biomass is carried out as a batch or continuous process.

Physical pre-treatment techniques include various types of milling/comminution (reduction of particle size), irradiation, steaming/steam explosion, and hydrothermolysis, in the preferred embodiment, soaking, removal of the solids from the liquid, steam exploding the solids to create the pre-treated ligno-cellulosic biomass.

Comminution includes dry, wet and vibratory ball milling. Preferably, physical pre-treatment involves use of high pressure and/or high temperature (steam explosion). In context of the invention high pressure includes pressure in the range from 3 to 6 MPa preferably 3.1 MPa. In context of the invention high temperature include temperatures in the range from about 100 to 300° C., preferably from about 160 to 235° C. In a specific embodiment impregnation is carried out at a pressure of about 3.1 MPa and at a temperature of about 235° C. In a preferred embodiment the physical pre-treatment is done according to the process described in WO 2010/113129, the entire teachings of which are incorporated by reference.

Although not needed or preferred, chemical pre-treatment techniques include acid, dilute acid, base, organic solvent, lime, ammonia, sulfur dioxide, carbon dioxide, pH-controlled hydrothermolysis, wet oxidation and solvent treatment.

If the chemical treatment process is an acid treatment process, it is more preferably, a continuous dilute or mild acid treatment, such as treatment with sulfuric acid, or another organic acid, such as acetic acid, citric acid, tartaric acid, succinic acid, or any mixture thereof. Other acids may also be used. Mild acid treatment means at least in the context of the invention that the treatment pH lies in the range from 1 to 5, preferably 1 to 3.

In a specific embodiment the acid concentration is in the range from 0.1 to 2.0% wt acid, preferably sulfuric acid. The acid is mixed or contacted with the ligno-cellulosic biomass and the mixture is held at a temperature in the range of around 160-220° C. for a period ranging from minutes to seconds. Specifically the pre-treatment conditions may be the following: 165-183° C., 3-12 minutes, 0.5-1.4% (w/w) acid concentration, 15-25, preferably around 20% (w/w) total solids concentration. Other contemplated methods are described in U.S. Pat. Nos. 4,880,473, 5,366,558, 5,188,673, 5,705,369 and 6,228,177.

Wet oxidation techniques involve the use of oxidizing agents, such as sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

Biological pre-treatment techniques include applying lignin-solubilizing micro-organisms (see, for example, Hsu, T.-A., 1996, Pre-treatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of ligno-cellulosic biomass, Adv. Appl. Microbiol. 39: 295-333; McMillan, J. D., 1994, Pretreating ligno-cellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of ligno-cellulosic hydrolysates for ethanol production, Enz. Microb. Tech. 18: 312-331; and Vallander, L., and Eriksson, K.-E. L., 1990, Production of ethanol from ligno-cellulosic materials: State of the art, Adv. Biochem. Eng./Biotechnol. 42: 63-95).

In an embodiment both chemical and physical pre-treatment is carried out including, for example, both mild acid treatment and high temperature and pressure treatment. The chemical and physical treatment may be carried out sequentially or simultaneously.

In a preferred embodiment the pre-treatment is carried out as a soaking step with water at greater than 1° C., removing the ligno-cellulosic biomass from the water, followed by a steam explosion step.

In a preferred embodiment the pre-treated ligno-cellulosic biomass is comprised of complex sugars, also known as glucans and xylans (cellulose and hemicellulose) and lignin.

The pre-treated ligno-cellulosic biomass may be subjected further to hydrolysis. Hydrolysis is conducted in the presence of a catalyst, or a catalyst composition. The catalyst may comprise at least a mineral acid and the hydrolysis is in this case an acid hydrolysis. Preferably, the catalyst comprises an enzyme or enzyme cocktail and the hydrolysis is an enzymatic hydrolysis.

The biomass will contain some compounds which are hydrolysable into a water soluble species obtainable from the hydrolysis of the biomass. In the case of water soluble hydrolyzed species of cellulose, cellulose can be hydrolyzed into glucose, cellobiose, and higher glucose polymers and includes dimers and oligomers. Thus some of the water soluble hydrolyzed species of cellulose are glucose, cellobiose, and higher glucose polymers and includes their respective dimers and oligomers. Cellulose is hydrolysed into glucose by the carbohydrolytic cellulases. Thus the carbohydrolytic cellulases are examples of catalysts for the hydrolysis of cellulose.

The prevalent understanding of the cellulolytic system divides the cellulases into three classes; exo-1,4-[beta]-D-glucanases or cellobiohydrolases (CBH) (EC 3.2.1.91), which cleave off cellobiose units from the ends of cellulose chains; endo-1,4-[beta]-D-glucanases (EG) (EC 3.2.1.4), which hydrolyse internal [beta]-1,4-glucosidic bonds randomly in the cellulose chain; 1,4-[beta]-D-glucosidase (EC 3.2.1.21), which hydrolyses cellobiose to glucose and also cleaves off glucose units from cellooligosaccharides. Therefore, if the biomass contains cellulose, then glucose is a water soluble hydrolyzed species obtainable from the hydrolysis of the biomass and the afore mentioned cellulases are specific examples, as well as those mentioned in the experimental section, of catalysts for the hydrolysis of cellulose.

By similar analysis, the hydrolysis products of hemicellulose are water soluble species obtainable from the hydrolysis of the biomass, assuming of course, that the biomass contains hemicellulose. Hemicellulose includes xylan, glucuronoxylan, arabinoxylan, glucomannan, and xyloglucan. The different sugars in hemicellulose are liberated by the hemicellulases. The hemicellulytic system is more complex than the cellulolytic system due to the heterologous nature of hemicellulose. The systems may involve among others, endo-1,4-[beta]-D-xylanases (EC 3.2.1.8), which hydrolyse internal bonds in the xylan chain; 1,4-[beta]-D-xylosidases (EC 3.2.1.37), which attack xylooligosaccharides from the non-reducing end and liberate xylose; endo-1,4-[beta]-D-mannanases (EC 3.2.1.78), which cleave internal bonds; 1,4-[beta]-D-mannosidases (EC 3.2.1.25), which cleave mannooligosaccharides to mannose. The side groups are removed by a number of enzymes; such as [alpha]-D-galactosidases (EC 3.2.1.22), [alpha]-L-arabinofuranosidases (EC 3.2.1.55), [alpha]-D-glucuronidases (EC 3.2.1.139), cinnamoyl esterases (EC 3.1.1.-), acetyl xylan esterases (EC 3.1.1.6) and feruloyl esterases (EC 3.1.1.73). Therefore, if the biomass contains hemicellulose, then xylose and mannose are examples of a water soluble hydrolyzed species obtainable from the hydrolysis of the hemicellulose containing biomass and the afore mentioned hemicellulases are specific examples, as well as those mentioned in the experimental section, of catalysts for the hydrolysis of hemicellulose.

Included in the hydrolysis process is a catalyst. The catalyst composition consists of the catalyst, the carrier, and other additives/ingredients used to introduce the catalyst to the process. As discussed above, the catalyst may comprise at least one enzyme or microorganism which converts at least one of the compounds in the biomass to a compound or compounds of lower molecular weight, down to, and including, the basic sugar or carbohydrate used to make the compound in the biomass. The enzymes capable of doing this for the various polysaccharides such as cellulose, hemicellulose, and starch are well known in the art and would include those not invented yet.

The catalyst composition may also comprise an inorganic acid preferably selected from the group consisting of sulfuric acid, hydrochloric acid, phosphoric acid, and the like, or mixtures thereof. The inorganic acid is believed useful for processing at temperatures greater than 100° C. The process may also be run specifically without the addition of an inorganic acid.

It is typical to add the catalyst to the process with a carrier, such as water or an organic based biomass. For mass balance purposes, the term catalyst composition therefore includes the catalyst(s) plus the carrier(s) used to add the catalyst(s) to the process. If a pH buffer is added with the catalyst, then it is part of the catalyst composition as well. Often the ligno-cellulosic biomass will contain starch. The more important enzymes for use in starch hydrolysis are alpha-amylases (1,4-[alpha]-D-glucan glucanohydrolases, (EC 3.2.1.1)). These are endo-acting hydrolases which cleave 1,4-[alpha]-D-glucosidic bonds and can bypass but cannot hydrolyse 1,6-[alpha]-D-glucosidic branchpoints. However, also exo-acting glycoamylases such as beta-amylase (EC 3.2.1.2) and pullulanase (EC 3.2.1.41) can be used for starch hydrolysis. The result of starch hydrolysis is primarily glucose, maltose, maltotriose, [alpha]-dextrin and varying amounts of oligosaccharides. When the starch-based hydrolysate is used for fermentation it can be advantageous to add proteolytic enzymes. Such enzymes may prevent flocculation of the microorganism and may generate amino acids available to the microorganism. Therefore, if the biomass contains starch, then glucose, maltose, maltotriose, [alpha]-dextrin and oligosaccharides are examples of a water soluble hydrolyzed species obtainable from the hydrolysis of the starch containing biomass and the afore mentioned alpha-amylases are specific examples, as well as those mentioned in the experimental section, of catalysts for the hydrolysis of starch.

### EXPERIMENTAL

### Example 1

### Screening Protocol Utilized for Fishing Plant Genes Conferring Increased Biochemical Stress Resistance

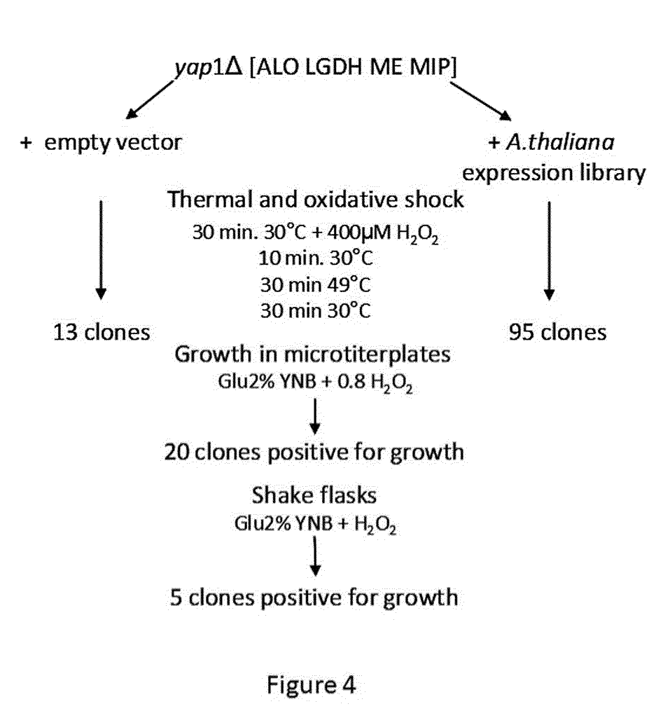

A screening procedure aimed at fishing A. thaliana genes able to increase the resistance of S. cerevisiae cells to biochemical stress was established. The screening procedure is reported in FIG. 4.

The screening procedure utilized as genetic background the S. cerevisiae strain BY4742Δyap1 [(MATa; ura3Δ0; his3Δ1; leu2Δ0; lys2Δ0; cir+), EuroScarf Accession No.YML007w (http://www.rz.uni-frankfurt.de/FB/fb16/mikro/euro scarf)]. Said Δyap1 mutant strain resulted per se hypersensitive to hydroperoxides because of the deficiency in the YAP1 transcription regulator (Evans et al. 1998, Kuge and Jones, 1994), rendering the setting of the stressful conditions for the screening easier to establish. Said strain has been engineered for the production of L-Ascorbic acid (afterwards referred to as BY4742Δyap1 L-AA producing). Details regarding the construction of the ascorbic acid producing strain has been disclosed in Branduardi et al., 2007 and here briefly reported. Template DNA for AtLGDH, AtME, and AtMIP was a cDNA library from A. thaliana (ATCC 77500); template DNA for ScALO1 consisted of 50 ng of genomic DNA from S. cerevisiae GRF18U (MAT; ura3; leu2-3,112; his3-11,15; cir+; Brambilla et al., 1999), extracted with a standard method (according to Hoffman and Winston, 1987, slightly modified). The following primer pairs were used: for ALO1 amplification: ALO1 for TTT CAC CAT ATG TCT ACT ATC C and ALO1rev AAG GAT CCT AGT CGG ACA ACT C; for LGDH amplification: LGDHfor ATG ACG AAA ATA GAG CTT CGA GC and LGDHrev TTA GTT CTG ATG GAT TCC ACT TGG; for ME amplification: MEfor GCG CCA TGG GAA CTA CCA ATG GAA CA and MErev GCG CTC GAG TCA CTC TTT TCC ATC A; for MIP amplification: MIP for ATC CAT GGC GGA CAA TGA TTC TC and MIPrev AAT CAT GCC CCT GTA AGC CGC. The PCR fragments were sub-cloned into the pSTBlue-1 vector using the Perfectly Blunt Cloning kit (Novagen) and checked by sequence analysis. The obtained sequences were the same as those reported in Genebank except for MIP in which two silent point substitutions (A271T and T685G) were detected. Finally, the coding sequences were EcoRI cut and sub-cloned into S. cerevisiae expression vectors of the YX series (R&D Systems, Inc.) or derivates (pYX012bT and pYX062, generated for the previously cited study, Branduardi et al., 2007) and into the centromeric expression vectors pZ₃ and pZ₅ (generated for the previously cited study, Branduardi et al., 2007). In detail, ALO1 was sub-cloned into pYX042 (integrative, LEU2 auxotrophic marker, ScTPI promoter); LGDH was sub-cloned into pYX022 (integrative, HIS3 auxotrophic marker, ScTPI promoter), ME into pYX062 (integrative, LYS2 auxotrophic marker, PCR amplified with oligonucleotides LYS2 for TGC CAG CGG AAT TCC ACT TGC and LYS2rev AAT CTT TGT GAA GCT TCG CAA GTA TTC ATT from S. cerevisiae genome and substituted to the URA3 auxotrophic marker in the plasmid pYX012 DraIII/NotI cut and blunt ended, ScTPI promoter) and MIP into pZ₅. Plasmid construction is described in subsequent experiment description.

The presence of the heterologous genes for the production of ascorbic acid was confirmed by PCR analysis. For each set of transformation at least three independent clones were initially tested, showing no meaningful differences among them.

The BY4742Δyap1 L-AA producing strain was transformed with the A. thaliana cDNA expression library constructed in the yeast multicopy expression vector pFL-61 (Minet et al., 1992; ATCC 77500), afterwards referred to as the “library transformed strain” and, in parallel, with the empty plasmid, afterwards referred to as the “control strain”. The transformation was executed according to the LiAc/PEG/SS-DNA protocol (Gietz & Woods 2002). A. thaliana cDNA manipulation, transformation and cultivation were performed in E. coli (Novablue, Novagen), following standard protocols (Sambrook J., et al., Molecular Cloning: A Laboratory Manual, 2^{nd} edn., Cold Spring Harbor Laboratory, New York, 1989). Also other basic molecular biology protocols were performed following this manual if not otherwise stated. All the restriction and modification enzymes utilised are from NEB (New England Biolabs, UK) or from Roche Diagnostics.

The transformants were plated on selective minimal medium and single colonies were obtained after a time ranging from 3 to 4 days of growth at 30° C., depending on colonies growth. Yeast cultures were grown in minimal synthetic medium (0.67% wv⁻¹ YNB Biolife without amino acids) with 2% wv-1 of glucose as carbon source. When required, based on the initial genome and the markers of the harboured expression vector(s), supplements such as leucine, uracil, lysine and histidine were added to a final concentration of 50 mg/l and the antibiotic nourseotricine sulphate (cloNAT, WERNER BioAgents, Germany) was added to a final concentration of 100 mg/l.

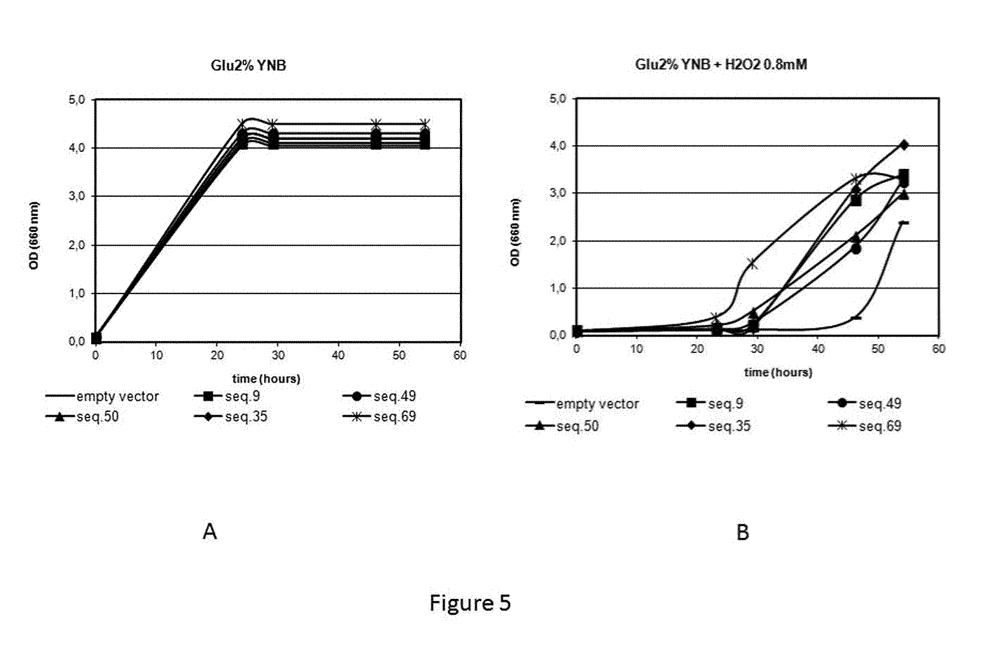

It was established a protocol for screening the resistance of cells to biochemical stress. To this purpose transformants, grown in liquid medium, were subjected to a pulse of heat and biochemical shock, as coupling these stresses is supposed to have an additive effect on ROS (Reactive Oxygen Species) accumulation, leading to increased cell death (Moraitis et al., 2004); cells (both control and library transformed strains) were then plated on glucose (URA-) selective medium and surviving colonies were counted after incubation for 3 days at 30° C. The pulse of biochemical and thermal shock was executed as follows: 30′ in shake flasks with minimal glucose medium at 30° C., under orbital shaking, with the addition in the medium of 400 μM H₂O₂; 10′ at 30° C. in fresh medium; 30′ at 49° C.; 30′ of recovery at 30° C. The 95 clones resulting from the library transformed strain and 13 clones obtained from the control strain were subjected to two successive rounds of tests aimed (i) to eliminate false positives and (ii) to identify clones able to exhibit enhanced tolerance not only under a pulse of stress, but also under biochemical stress conditions. To this purpose the 95 transformants and the 13 control clones were subjected to a primary test in microtiter plates in the presence of H₂O₂ 0.7 mM, and their growth was measured at regular intervals of time over 72 hours. In these conditions 20 out of the 95 transformants were able to grow earlier than the others (including the controls). Growth was measured by optical density at 660 nm; initial optical density was 0.015 in 200 μl of glucose minimal medium. The secondary test was performed in batch, shake-flasks, increasing H₂O₂ to 0.8 mM (and inoculating cells in a total volume of 20 ml, at an initial optical density of 0.1). This test further reduced the number of promising clones to 5. These 5 clones (numbered as 9, 35, 49, 50 and 69), grew faster than the control ones, exhibiting enhanced tolerance to H₂O₂, as evidenced in FIG. 5B. All clones grew at a comparable level in the absence of H₂O₂, as evidenced in FIG. 5A, demonstrating that there were no evident defects of growth as a consequence of the expression of the exogenous genes.

### Example 2

Identification of the plant sequence contained in transformant no 69 and effects of its (over)expression in the S. cerevisiae GRF18U strain in respect to biochemical and acidic stress.

Plasmids containing the cDNA of the A. thaliana library were successively rescued from these yeast clones. The corresponding A. thaliana sequences were PCR amplified from said plasmids used as template. The following primers were respectively drawn on the PGKS and PGK3 sequences of the PFL61 plasmid (Minet et al., 1992):

AraBankFOR :
5′-AAA CTT ACA TTT ACA TAT ATA TAA ACT TGC-3′ → 55.8° C.
AraBankREV:
5′-GTA TAT AAA TAA AAA ATA TTC AAA AAA TAA AAT AAA CTA T-3′ → 56.1° C.

The PCR amplified products were subsequently sub-cloned into pSTBlue-1 vector using the Perfectly Blunt Cloning kit (Novagen), and the resulting plasmids were sequenced for the plant inserts. Sequencing analyses revealed that the original library transformant assigned with no 69 was transformed with a partial PAL3 sequence. The partial PAL3 nucleotidic sequence encodes the aminoacidic sequence SEQ ID NO: 1 reported in FIG. 2, bearing the C-terminal portion of the protein (417 aminoacidic residues out of 694 total aminoacidic residues of PAL3 (AT5G04230). The total PAL3 nucleotidic sequence encodes the aminoacidic sequence SEQ ID NO: 2 reported in FIG. 3. The corresponding shuttle plasmid constructed for sequencing was identified as “pSTBlue-1 AtPAL3partial” vector.

In order to prove the ability of the selected cDNA to confer tolerance to biochemical stress not only in a mutant strain, per se hypersensitive to this conditions, but also in wild type yeasts, the partial PAL3 coding sequence and the complete PAL3 coding sequence, from A. thaliana were newly cloned in opportune expression vectors which were used to transform the S. cerevisiae strain GRF18U (MATa; ura3; leu2-3,112; his3-11,15; cir+; Brambilla et al., 1999).

First of all the complete sequence of AtPAL3 was PCR amplified. The A. thaliana expression library (ATCC 77500) was used as template with the following primers:

AtPAL3FOR :
5′-ATG GAG TTT CGT CAA CCA AAC-3′ → 55.9° C.
AtPAL3REV :
5′-TTA GCA GAT AGA AAT CGG AGC A- → 56.5° C.

The resulting PCR fragment was sub-cloned into pStBlue-1 vector using the Perfectly Blunt Cloning kit (Novagen), resulting in the pSTBlue-1 AtPAL3total vector, and sequenced. The obtained sequence was the same as that reported in Genebank (AT5G04230) except for three substitutions (positions 269, 1464, 1879), resulting in only two aminoacidic substitutions (position 91 D→G and position 627 A→T).

The PAL3 partial and complete sequences were then EcoRI cut from the respective E. coli shuttle vector previously generated (pSTBlue-1 AtPAL3partial and pSTBlue-1 AtPAL3total, respectively) and cloned into the GRF18U yeast expression vector pZ₅ EcoRI cut and dephosphorylated to obtain the “pZ₅PAL3total” and “pZ₅PAL3partial” vectors. PZ₅ is a centromeric vector bearing the nourseotricine cassette (Nat^{R}) as antibiotic selection. It is derived from pBR1, which in turn is a pYX022 (R&D Systems, Inc.)-derivative in which the ARS-CEN fragment from Ycplac33 has been inserted (Branduardi et al, 2004). The Nat^{R} cassette PvuII/SacI cut and blunt ended from pAG25 (Goldstein & McCusker, 2009) has been inserted into pBR1 cut KpnI and blunt ended, to obtain pZ5 vector (Branduardi et al., 2007). The resulting plasmids were used to transform the strain GRF18U according to the LiAc/PEG/ss-DNA protocol (Gietz & Woods 2002).

For growth in shake flasks strains were inoculated at an initial optical density of 0.1 (660 nm) in 20 ml of the opportune medium (based on the initial genome and the markers of the harboured expression vector(s) at 30° C. and 160 rpm and the ratio of flask volume/medium was of 5/1.

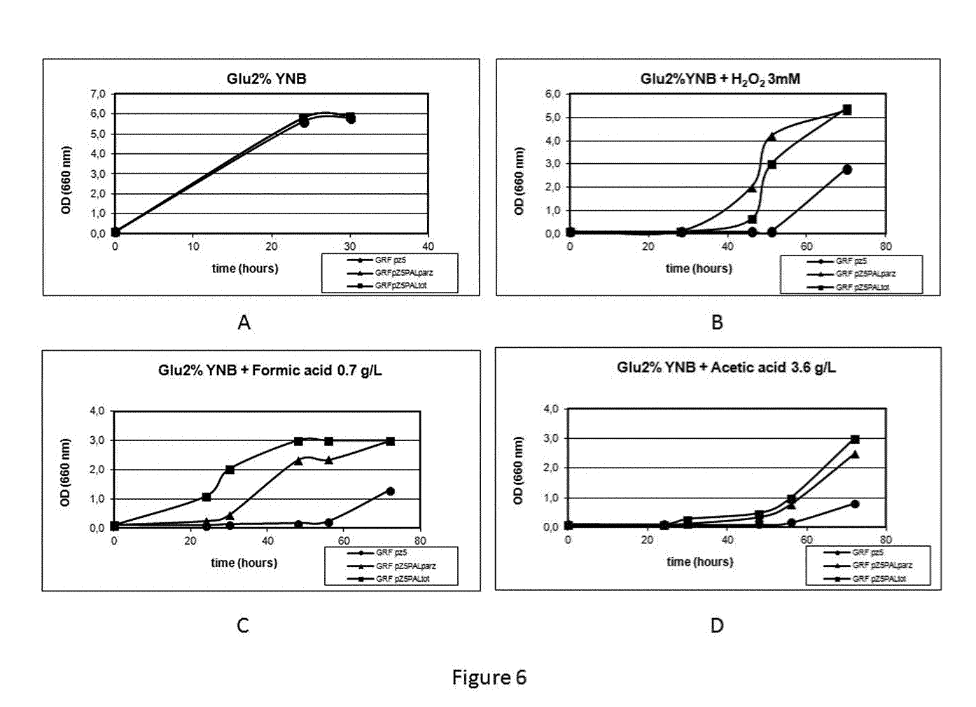

Remarkably, even if the PAL3 sequence identified through the screening lacked the N-terminal portion, being composed by amino-acids from 278 to 694 (end of the protein), it was nevertheless able to increase tolerance to biochemical stress driven by 3 mM H₂O₂, as clearly shown in FIG. 6B; the expression of the PAL3 complete sequence influenced even more positively the growth of the respective transformant strain. In FIG. 6A is reported the graph of the growth in the absence of stress agents.

The acquired resistance was not only against biochemical stress, but also against organic acid stress, as demonstrated by FIG. 6C and FIG. 6D. Engineered and control strains were grown in minimal medium in the presence of increasing concentrations of formic (15 mM) or acetic acid (60 mM) at pH 3 (to maintain their undissociated form; pka=3.75 and 4.74 for formic and acetic acid, respectively). Both strains expressing a partial and complete PAL3 sequence exhibited an increased stress resistance; moreover, as the case of H₂O₂, this feature was more pronounced in the GRF18Uc [PAL3 total] strain (GRF18Uc corresponds to the GRF18U strain cured for uracil, histidine and leucine auxotrophy by transforming it with empty yeast vectors containing the URA3, HIS3 and LEU2 genes).

Cells grown under stress by the addition of H₂O₂ 3 mM were subjected to cytofluorimetic analysis to deeper investigate the acquired stress tolerance conferred by the expression of PAL3.

Flow cytometric analyses were performed as described in Branduardi et al., 2007. Briefly, cells were stained with Dihydrorhodamine123 (DHR123), washed twice with PBS buffer and then resuspended in propidium iodide solution 0.46 mM (double staining). Alternatively an equal number of cells were stained with DHR123 or propidium iodide (PI, single staining). A sample of cells exponentially growing in optimal conditions was similarly treated and considered as “negative standard” (i.e., a sample of cells negative for both staining). Similarly, a sample was treated for 20 minutes at −20° C. with ice-chilled 70% ethanol for killing all the cells and resulting in a PI positive control (i.e., almost 100% PI positive cells). Finally, a sample was treated with H2O2 10 mM for 30′, resulting in a ROS positive control (i.e., sample of cells almost 100% positive to the DHR123 staining). Samples were then analysed using a Cell Lab Quanta™ SC flow cytometer (Beckman Coulter, Fullerton, Calif., USA). Excitation wavelength is at 488 nm, the laser power was settled at 22 mw; FL1 channel registers the emission of the esterified DHR123 (peak at 534 nm) while FL3 channel registers the emission of the PI (peak at 617 nm). The sample flow rate during analysis did not exceed 600-700 cells/s. A total of 20,000 cells were measured for each sample. Data analysis was performed afterwards with WinMDI 2.8 software, build#13 01-19-2000 (Purdue University, Cytometry Laboratories [http://facs.scripps.edu/software.html]).

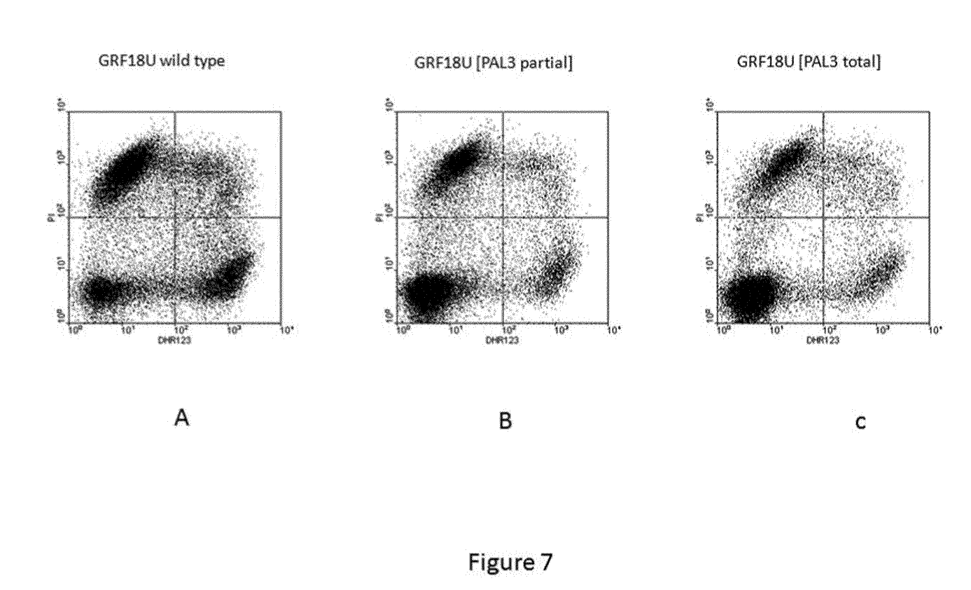

At the time in which all strains started to recover from the imposed stress, corresponding to cellular growth that can be visualised by a slight increase in optical density (OD) at 660 nm, samples were taken (more precisely, samples were taken when OD660 nm, starting from an initial value of 0.1, raised up to 0.11-0.12). Each sample was double stained with dihydrorhodamine 123, for the detection of Reactive Oxygen Species (Madeo et al., 1999), and with PI (propidium iodide) to detect severely damaged/dead cells (Sasaki et al., 1987); the resulting dot plots were subsequently compared (FIG. 7). FIG. 7A relates to control strain: under this condition, a considerable fraction of the analysed cells (22%) displayed high ROS content (high rhodamine signal, for convenience indicated as DHR123 in figures) and the proportion of dead cells (43%, high PI signal) largely exceeded that of viable cells (24%, low rhodamine and PI signal). By contrast, in the strain engineered with partial PAL3 sequence (FIG. 7B), a clear reduction in the ROS content (8% of the analysed population) and a significant increase in cell viability (60%) were registered. Remarkably, this trend was maximised in GRF18Uc [PAL3tot] (FIG. 7C), were only 18% of dead cells and 5% cells with high ROS content were detected, while almost 72% of the analysed cells were still healthy. In Each subpopulation was determined by drawing on the dot plot axes according to the distribution obtained with positive and negative samples (see Methods above). Data are summarised in Table 1.

TABLE 1
Percentages of cells with high ROS signal and/or high PI signal
for GRF18U wild type, GRF18U [PAL3 partial] and
GRF18U [PAL3 total] as grown in H2O2.

| | GRF18U | GRF18U | GRF18U |
|---|---|---|---|
| | wild type | [PAL3 partial] | [PAL3 total] |
| A subpopulation | 24.12% | 59.78% | 71.72% |
| B subpopulation | 22.16% | 8.55% | 5.38% |
| C subpopulation | 10.28% | 4.73% | 5.04% |
| D subpopulation | 43.44% | 26.94% | 17.95% |

Similar results were obtained analysing strains grown under formic or acetic acid treatment, since also in these conditions proportion of dead cells and of cells with high ROS content were decreased by the heterologous A. thaliana activities.

Taken together these data indicate that PAL3 both in its partial and total sequence, protects, directly or indirectly, cells from various stresses, contributing to scavenge ROS content and increasing cell viability. Viability is a crucial parameter whenever a microbial cell is utilized for a process of production, especially when the product derives from its central metabolism, as is the case for the ethanol production. Viability is the main parameter used for determining the increased resistance to biochemical stress. When colonies of two different microbial cells have viability values which are different for less than 3%, still viable but ROS accumulating cells (low PI, high rhodamine signal) is taken as the secondary parameter used for evaluating the resistance to biochemical stress.

### Example 3

(Over)expression of the A. thaliana PAL3 complete coding sequence in the S. cerevisiae industrial strains AP (Arome Plus, purchased by AEB group, Italy) and VIN13 (purchased by Anchor, France): effect of acetic acid stress and ethanol production and productivity.

The PAL3 complete sequence was expressed in the industrial S. cerevisiae strains (VIN13 and AP) and tested for acetic acid tolerance and for ethanol production.

PAL3 complete sequence was EcoRI cut from the respective E. coli shuttle vector previously generated (pSTBlue-1 AtPAL3total) and cloned into the yeast expression vector p012NAT EcoRI cut and dephosphorylated, resulting in the plasmid p012NATPAL.

P012NAT derives from the basic S. cerevisiae integrative expression plasmid pYX012 (R&D Systems, Inc., Wiesbaden, D), which harbour the ScTPI promoter for leading gene expression and the auxotrophic URA3 marker for transformants selection. Because industrial strains are prototrophic, a cassette conferring resistance to the antibiotic nourseotricin was added to the described basic plasmid. Said cassette was EcoRV/PvuII cut from pAG25 (Goldstein and McCusker, 1999) and inserted into pYX012 KpnI cut, blunt ended and dephosphorylated.

p012NATPAL was used to transform the above mentioned industrial and laboratory strains. Transformants were selected on YEPD agarose plates with rich glucose medium, added with the antibiotic nourseotricine sulphate (cloNAT, WERNER BioAgents, Germany) added to a final concentration of 100 mg/L. Positive clones were PCR verified.

The resulting transformants, together with transformants bearing the sole empty plasmid and the original strain, not transformed, were shake-flask cultured in medium added with different concentrations of acetic acid, comparing the different growth curves. More in detail, Inhibition tests were performed in 250 ml flask filled with 100 ml of YEPD medium at pH 5, with an initial glucose concentration of 4% (40 g/L), at 32° C., and 200 rpm, inoculated at OD 660 nm=0.6.

YEPD medium formulation:

20 g/L Peptone (cod. P7750 Sigma-Aldrich)

10 g/L Yeast extract (cod. 70161 Sigma-Aldrich)

Sterilized in autoclave for 20 min at 121° C.

Adjust the pH at 5 with KOH 2M sterile solution.

Growth kinetics was measured in YEPD medium, with glucose 4% (w/V) as a carbon source added with acetic acid 7.5 and 11.25 g/L, respectively for VIN13 and AP strains. Growth was measured over time as optical density (OD 660 nm) for wild type strains, strains transformed with the sole control plasmid, and the strains bearing the total AtPAL3 coding sequence. The data correspond to the mean values of three independent biological replicates. Standard error is lower than 0.03%. Wild type strains and strains transformed with the sole control plasmid have the save growth kinetics both for VIN13 and AP strains.

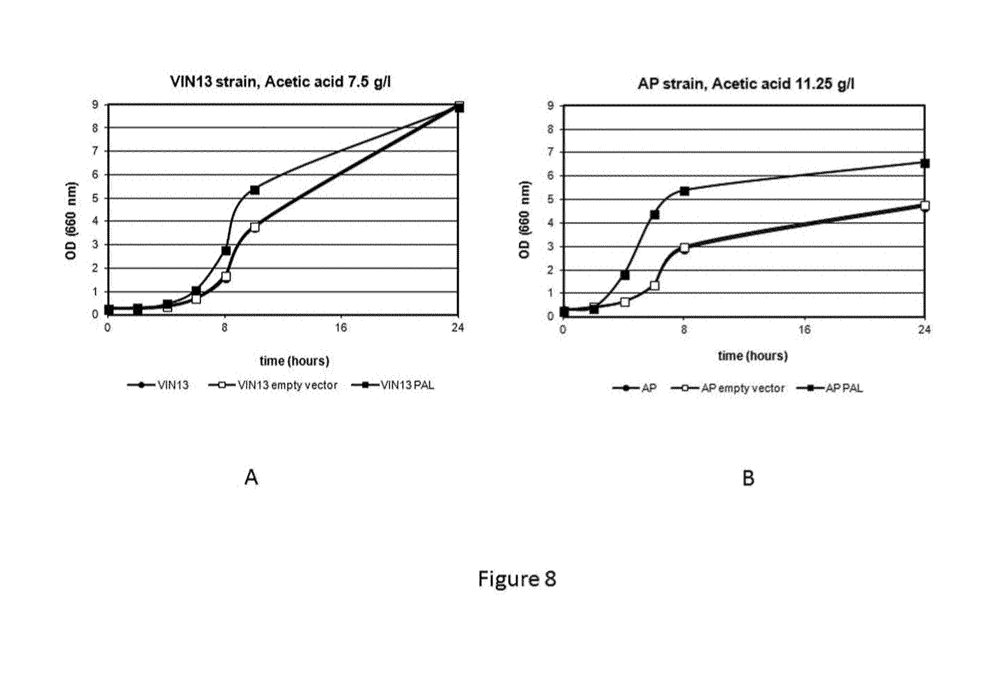

In all the tested strains, whenever the acetic acid concentration started to become limiting for growth (i.e., increasing the time of lag-phase), the clones bearing the plant PAL3 genes resulted advantaged in rescuing the growth, as illustrated In FIG. 8A for the strain VIN13 grown in medium added with 7.5 g/L of acetic acid. Remarkably, the advantage derived from the expression of the PAL3 gene remains evident for the strain background AP even when 11.25 g/L of acetic acid are added to the medium, as represented in FIG. 8B.

Finally, the AP strain bearing or not the plant PAL3 gene was tested in bioreactor for ethanol production. Tests were performed in a bioreactor filled with 1.5 l of Verduin medium at pH 5, with an initial glucose concentration of 140 g/L, at 30° C., and 250 rpm, with initial inoculum at OD 660 nm=1.

Medium formulation (Verduin medium)

150 g/l Glu

5 g/l (NH4)2SO4

3 g/l KH2PO4

0.5 g/l MgSO4 7H2O

Acetic Acid 10 g/L

Medium was sterilized in autoclave for 20 min at 121° C. and adjusted for an initial pH at 5 with KOH 2M sterile solution.

Initial inoculum was performed at OD660ₙₘ of 0.6.

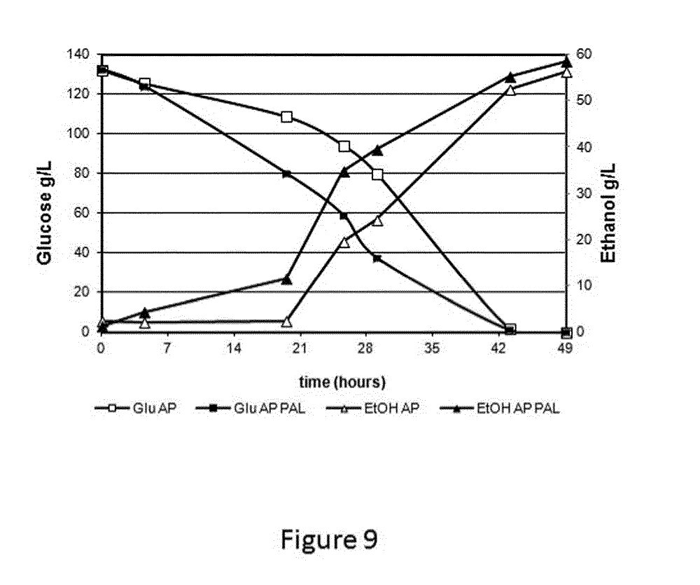

FIG. 9 reports the glucose concentration and ethanol concentration of the modified and the not modified AP strain.

Remarkably, in the strain expressing PAL3, the ethanol production is significantly anticipated, with a positive effect on productivity.

### REFERENCES

Abbott et al. (2009) “Catalase overexpression reduces lactic acid-induced biochemical stress in Saccharomyces cerevisiae.” Appl Environ Microbiol. 75(8):2320-5.

Brambilla L, Bolzani D, Compagno C, Carrera V, van Dijken J P, et al. (1999) NADH reoxidation does not control glycolytic flux during exposure of respiring Saccharomyces cerevisiae cultures to glucose excess. FEMS Microbiol Lett 171: 133-140.

Branduardi P, Valli M, Brambilla L, Sauer M, Alberghina L, et al. (2004) The yeast Zygosaccharomyces bailii: a new host for heterologous protein production, secretion and for metabolic engineering applications. FEMS Yeast Res 4: 493-504.

Branduardi P., Fossati T., Sauer M., Pagani R., Mattanovich D., Porro D. (2007) Biosynthesis of vitamin C by yeast leads to increased stress resistance. PlosS ONE 2(10): e1092

Du et al. (2008) “Formic acid induces Ycalp-independent apoptosis-like cell death in the yeast Saccharomyces cerevisiae.l ” FEMS Yeast Res. 8(4): 531-9.

Evans M V, Turton H E, Grant C M, Dawes I W (1998) J. Bacteriol. 180 483-490

Gietz R D, & Woods R A (2002) Transformation of yeast by lithium acetate/single-stranded carrier DNA/polyethylene glycol method. Methods Enzymol 350: 87-96.

Goldstein A L, McCusker J H (1999) Three new dominant drug resistance cassettes for gene disruption in Saccharomyces cerevisiae. Yeast 15: 1541-1553.

Hoffman C S, Winston F (1987) A ten-minute DNA preparation from yeast efficiently releases autonomous plasmids for transformation of Escherichia coli. Gene 57: 267-272.

Kim et al., (2012) “Rice ASR1 protein with reactive oxygen species scavenging and chaperone-like activities enhances acquired tolerance to abiotic stresses in Saccharomyces cerevisiae.” Mol Cells 33(3):285-93.

Kuge S and Jones N (1994) EMBO J 13 Madeo F, Frohlich E, Ligr M, Grey M, Sigrist S J, et al. (1999) Oxygen stress: a regulator of apoptosis in yeast. J Cell Biol 145: 757-767

Minet M, Dufour M E, Lacroute F (1992) Complementation of Saccharomyces cerevisiae auxotrophic mutants by Arabidopsis thaliana cDNAs. Plant J 2: 417-422.

Moraitis et al. (2004) Reactive oxygen species may influence the heat shock response and stress tolerance in the yeast Saccharomyces cerevisiae Piper et al., (1999) “Yeast superoxide dismutase mutants reveal a pro-oxidant action of weak organic acid food preservatives.” Free Radic Biol Med. 27: 1219-27

Sasaki D T, Dumas S E, Engleman E G (1987) Discrimination of viable and non-viable cells using propidium iodide in two color immunofluorescence. Cytometry 8: 413-420.

Semchyshyn et al., 2011) “Acetate but not propionate induces biochemical stress in bakers' yeast Saccharomyces cerevisiae.” Redox Rep. 16(1):15-23.

Valko M, Rhodes C J, Moncol J, Izakovic M, Mazur M (2006) Free radicals, metals and antioxidants in oxidative stress-induced cancer. Chem Biol Interact 160: 1-40.

Yang et al., (2012) “Ethanol reduces mitochondrial membrane integrity and thereby impacts carbon metabolism of Saccharomyces cerevisiae.” FEMS Yeast Res. 12(6):675-84.

We claim:

1-38. (canceled)

39. A non-naturally occurring microbial organism, derived from a starting microbial organism, wherein
a) the non-naturally occurring microbial organism comprises at least one exogenous nucleic acid encoding an enzyme, or a portion thereof, selected from the group of ammonia, lyase, and
b) the non-naturally occurring microbial organism has an increased resistance to biochemical stress compared to the starting microbial organism.

40. The non-naturally occurring microbial organism according to claim 39, wherein the enzyme is selected from the group consisting of EC.4.3.1.X, where X is an integer from 1 to 27 inclusive.

41. The non-naturally occurring microbial organism according to claim 40, wherein the enzyme is selected from the group consisting of EC.4.3.1.23, EC.4.3.1.24, EC.4.3.1.25, and EC.4.3.1.3.

42. The non-naturally occurring microbial organism according to claim 39, wherein the enzyme is selected from the group consisting of phenylalanine ammonia-lyase (PAL), tyrosine ammonia-lyase (TAL), phenylalanine/tyrosine ammonia-lyase (PAL/TAL), and histidine ammonia-lyase (HAL).

43. The non-naturally occurring microbial organism according to claim 42, wherein the enzyme is PAL.

44. The non-naturally occurring microbial organism according to claim 43, wherein the enzyme is PAL3.

45. The non-naturally occurring microbial organism according to claim 39, wherein the at least one exogeneous nucleic acid is obtained from a plant.

46. The non-naturally occurring microbial organism according to claim 45, wherein the at least one exogeneous nucleic acid is obtained from Arabidopsis thaliana.

47. The non-naturally occurring microbial organism according to claim 39, wherein the enzyme comprises SEQ ID NO: 1 or an amino acid sequence having a sequence identity to SEQ ID NO: 1 selected from the group consisting of at least 98%, at least 95%, at least 90%, at least 80%, at least 70%, at least 60%, at least 50%, and at least 40%.

48. The non-naturally occurring microbial organism according to claim 39, wherein the enzyme is SEQ ID NO: 2 or an amino acid sequence having a sequence identity to SEQ ID NO:2 selected from the group consisting of at least 98%, at least 95%, at least 90%, at least 80%, at least 70%, at least 60%, at least 50%, and at least 40%.

49. The non-naturally occurring microbial organism according to claim 39, wherein the enzyme is a fragment of SEQ ID NO: 2.

50. The non-naturally occurring microbial organism according to claim 39, wherein the biochemical stress is induced by at least one of a reactive oxygen species, an organic acid, a mineral acid, or an alcohol.

51. The non-naturally occurring microbial organism according to claim 39, wherein the increased resistance to biochemical stress compared to the starting microbial organism is indicated by at least one of an increase in the percentage of viable cells or a decrease in the amount of reactive oxygen species (ROS), under the same condition.

52. The non-naturally occurring microbial organism according to claim 39, wherein the stress is induced by ethanol and/or acetic acid.

53. The non-naturally occurring microbial organism according to claim 39, wherein the enzyme catalyzes a reaction to produce at least a cinnamic acid.

54. The non-naturally occurring microbial organism according to claim 53, where in the at least a cinnamic acid is selected from the group consisting of cis-cinnamic acid, trans-cinnamic acid and para-hydroxy cinnamic acid.

55. The non-naturally occurring microbial organism according to claim 53, wherein the at least a cinnamic acid comprises cis-cinnamic acid.

56. The non-naturally occurring microbial organism according to claim 53, wherein the at least a cinnamic acid comprises trans-cinnamic acid.

57. The non-naturally occurring microbial organism according to claim 53, wherein the at least a cinnamic acid comprises parahydroxy cinnamic acid.

58. The non-naturally occurring microbial organism according to claim 39, wherein the starting microbial organism is a non-naturally occurring microbial organism.

59. The non-naturally occurring microbial organism according to claim 39, wherein the starting microbial organism is selected from the group consisting of yeasts, bacteria, fungi.

60. The non-naturally occurring microbial organism according to claim 59, wherein the starting microbial organism is a yeast.

61. The non-naturally occurring microbial organism according to claim 60, wherein the yeast is selected from the group consisting of Saccharomyces, Candida, Zygosaccharomyces, Hansenula, Kluyvoremyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, and Schwanniomyces.

62. The non-naturally occurring microbial organism according to claim 61, wherein the yeast is Saccharomyces cerevisiae yeast.

63. The non-naturally occurring microbial organism according to claim 62, wherein the strain of Saccharomyces cerevisiae yeast is selected from the group consisting of GRF18U, BY4742, CEN.PK strains 102-5B and 113-11C, VIN13, and AP.

64. The non-naturally occurring microbial organism according to claim 59, wherein the starting microbial organism is a fungus.

65. The non-naturally occurring microbial organism according to claim 59, wherein the starting microbial organism is a bacterium.

66. The non-naturally occurring microbial organism according to claim 65, wherein the bacterium is selected from the group consisting of Escherichia coli, Klebsiella oxytoca, Anaerobiospirillum succiniciprodiicens, Actino bacillus succinogenes, Mannheimia succiniciprodiicens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicu, Pseudomonas fluorescens, and Pseudomonas putida.

67. A method for producing a product, comprising culturing the non-naturally occurring microbial organism according to claim 39 in a culture medium comprising a carbon source, under conditions and for a sufficient period of time to produce the product.

68. The method according to claim 67, wherein the non-naturally occurring microbial organism is cultured in the presence of at least on biochemical stress agent.

69. The method according to claim 68, wherein the at least one biochemical stress agent is selected from the group consisting of acetic acid, formic acid, hydrogen peroxide, and ethanol.

70. The method according to claim 68, wherein the at least one biochemical tress agent comprises acetic acid.

71. The method according to claim 70, wherein the concentration of acetic acid is selected from the group consisting of higher than 3.5 g/l, higher than 7 g/l, higher than 11 g/l, and higher than 15 g/l.

72. The method according to claim 67, wherein at least a fraction of the carbon source is obtained from a ligno-cellulosic feedstock.

73. The method according to claim 72, wherein the ligno-cellulosic feedstock is subjected to a pretreatment to produce a pretreated ligno-cellulosic feedstock.

74. The method according to claim 72, wherein the at least a fraction of the pretreated ligno-cellulosic feedstock is subjected to a hydrolysis process.

75. The method according to claim 74, wherein the hydrolysis process is an enzymatic hydrolysis.

76. The method according to claim 67, wherein the product comprises ethanol.


SEQUENCE LISTING (2 sequences)

SEQ ID NO: 1 - protein, 423 aa, Saccharomyces cerevisiae
EVASAMFAEVMQGKPEFTDHLTHKLKHHPGQIEAAAIMEHILDGSSYVKEALHLHKIDPLQKPKQDRYALRTSPQWLGPQIEVIRAATKMIEREINSVNDNPLIDVSRNKAIHGGNFQGTPIGVAMDNTRLALASIGKLMFAQFTELVNDFYNNGLPSNLSGGRNPSLDYGLKGAEVAMASYCSELQFLANPVTNHVESASQHNQDVNSLGLISSRTTAEAVVILKLMSTTYLVALCQAFDLRHLEEILKKAVNEVVSHTAKSVLAIEPFRKHDDILGVVNREYVFSYVDDPSSLTNPLMQKLRHVLFDKALAEPEGETDTVFRKIGAFEAELKFLLPKEVERVRTEYENGTFNVANRIKKCRSYPLYRFVRNELETRLLTGEDVRSPGEDFDKVFRAISQGKLIDPLFECLKEWNGAPISIC

SEQ ID NO: 2 - protein, 694 aa, Saccharomyces cerevisiae
MEFRQPNATALSDPLNWNVAAEALKGSHLEEVKKMVKDYRKGTVQLGGETLTIGQVAAVASGGPTVELSEEARGGVKASSDWVMESMNRGTDTYGITTGFGSSSRRRTDQGAALQKELIRYLNAGIFATGNEDDDRSNTLPRPATRAAMLIRVNTLLQGYSGIRFEILEAITTLLNCKITPLLPLRGTITASGDLVPLSYIAGFLIGRPNSRSVGPSGEILTALEAFKLAGVSSFFELRPKEGLALVNGTAVGSALASTVLYDANILVVFSEVASAMFAEVMQGKPEFTDHLTHKLKHHPGQIEAAAIMEHILDGSSYVKEALHLHKIDPLQKPKQDRYALRTSPQWLGPQIEVIRAATKMIEREINSVNDNPLIDVSRNKAIHGGNFQGTPIGVAMDNTRLALASIGKLMFAQFTELVNDFYNNGLPSNLSGGRNPSLDYGLKGAEVAMASYCSELQFLANPVTNHVESASQHNQDVNSLGLISSRTTAEAVVILKLMSTTYLVALCQAFDLRHLEEILKKAVNEVVSHTAKSVLAIEPFRKHDDILGVVNREYVFSYVDDPSSLTNPLMQKLRHVLFDKALAEPEGETDTVFRKIGAFEAELKFLLPKEVERVRTEYENGTFNVTNRIKKCRSYPLYRFVRNELETRLLTGEDVRSPGEDFDKVFRAISQGKLIDPLFECLKEWNGAPISIC